(19) United States
(12) Patent Application Publication
(10) Pub. No.: US 2022/0023231 A1
(43) Pub. Date: Jan. 27, 2022

(54) METHOD OF TREATING POSTPRANDIAL INFLAMMATION AND PREVENTING WEIGHT GAIN

(71) Applicant: DASMAN DIABETES INSTITUTE, DASMAN (KW)

(72) Inventors: FATEMA AL-RASHED, DASMAN (KW); RASHEED AHMAD, DASMAN (KW)

(21) Appl. No.: 17/495,549

(22) Filed: Oct. 6, 2021

Related U.S. Application Data
(63) Continuation-in-part of application No. 16/783,775, filed on Feb. 6, 2020.

(51) Int. Cl. A61K 31/04; A61P 29/00; A61P 3/04

(52) U.S. Cl. CPC A61K 31/04; A61P 3/04; A61P 29/00

(57) ABSTRACT

A method of treating postprandial inflammation, or inhibiting an inflammatory response in a subject, may include administering to a subject an edible composition comprising an ACSL1 inhibitor. The ACSL1 inhibitor may be administered before or during consumption of a food high in fat, particularly a food high in saturated fat. The ACSL1 inhibitor may prevent a pro-inflammatory response, weight gain, or the development or progression of a disease associated with the consumption of high fat foods, such as dyslipidemia, obesity, diabetes, arthritis, or hepatic steatosis.

### CROSS-REFERENCE TO RELATED APPLICATIONS

This application is a continuation-in-part of U.S. patent application Ser. No. 16/783,775, filed on Feb. 6, 2020, presently pending.

### FIELD

The disclosure of the present patent application relates to formulations and methods useful in treating inflammation, inhibiting an inflammatory response, and preventing weight gain, and particularly to formulations and methods of reducing or regulating an inflammatory response due to diet, based on a long chain acyl-CoA synthetase (ACSL) inhibitor. These formulations may also be useful in preventing weight gain and development of associated diseases that normally result from eating a high fat diet.

### DESCRIPTION OF THE RELATED ART

Food items in contemporary diets are often high in saturated fatty acids (SFA), such as palmitic acid (PA). SFA are known to up-regulate inflammation upon consumption. Acute inflammatory responses can be initiated by food consumption, particularly high fat food consumption, such responses being called postprandial inflammation. Postprandial inflammation arises primarily from lipemia caused by increased chylomicron formation and triacylglyceride (TAG) content in circulation following food consumption. Consuming a meal high in SFA may cause particularly acute postprandial inflammatory effects, which could further cause or aggravate metabolic conditions, such as insulin resistance or diabetes, or existing inflammatory conditions, such as arthritis. In lieu of dietary intervention, a pharmaceutical intervention is desired to prevent or minimize postprandial inflammation.

Acyl CoA synthetases (ACSL), particularly ACSL1, are major enzymes responsible for converting free fatty acids taken in through diet into several lipid subclasses usable as energy sources, cellular building blocks or cellular communication means. Free fatty acids, such as SFA, can only be utilized by the body after their activation or catalyzation by Acyl-CoA. In particular, work by the present inventors has shown that Acyl-CoA activation is important for tumor necrosis factor alpha (TNF-α) signaling, TNF-α being a critical pro-inflammatory cytokine. The use of ACSL inhibitors was also shown to stop TNF-α signaling, thus stopping inflammatory responses in vitro (Al-Rashed, F., Ahmad, Z., Iskandar, M. A., Tuomilehto, J., Al-Mulla, F., & Ahmad, R. (Mar. 8, 2019). “TNF-α induces a pro-inflammatory phenotypic shift in monocytes through ACSL1: relevance to metabolic inflammation”. Cell Physiol Biochem, 52(3), 397-407). There are a limited number of TNF-α inhibitors available, and most become unusable because of tolerance within a year.

While dietary fatty acids are essential for several metabolic processes, certain saturated fatty acids, such as PA, induce macrophage foaming and pro-inflammatory responses. These damaging processes might be limited in normal weight individuals, but in obese subjects, the increased amount of adipose tissue acts as a constant fuel, triggering a sustained or extreme monocyte/macrophage pro-inflammatory response. This also triggers macrophages to infiltrate adipose tissue, producing obesity-related inflammation. A means of blocking bad fatty acids during high intake of free fatty acids would break this cycle and prevent further pro-inflammatory response and damage.

Previous work has focused on developing drugs to assist with weight loss rather than prevention of weight gain. Further, this previous work has focused on drugs that inhibit fatty acid synthase or other targets involved in the fatty acid synthetic pathway. (See for example US 2006/0241177 A1 and US 2009/0005435 A1)

Thus, a formulation and method of treating or preventing postprandial inflammation and preventing weight gain is desired.

### SUMMARY

A formulation for treating inflammation or inhibiting an inflammatory response according to the present disclosure includes a composition comprising an ACSL1 inhibitor or a pharmaceutically acceptable salt thereof. The composition, in one embodiment, can be an oral, or edible, composition. The edible composition may be a beverage or soluble additive to a beverage, and the beverage may be a carbonated beverage.

In one embodiment, the present subject matter relates to a method of treating postprandial inflammation, or inhibiting a postprandial inflammatory response, in a subject, comprising administering to a subject in need thereof a composition comprising an ACSL1 inhibitor or a pharmaceutically acceptable salt thereof in an amount effective to treat the postprandial inflammation or inhibit the postprandial inflammatory response. In an embodiment, the composition is a beverage administered orally within hours of the subject consuming a food. In an embodiment, the edible composition is administered by consumption by the subject and the subject consumes the edible composition before, at about the same time as, or after the subject consumes the food. The food may be high in SFA.

The composition may act as a neutralizer to acute effects of fatty acids ingested by the subject during consumption of food, such as fast food, rich in fatty acids, such as SFA, thereby preventing or minimizing the acute effects of said SFA.

In a further embodiment, the present subject matter relates to a method of preventing weight gain in a subject, comprising administering to a subject in need thereof an edible composition comprising an ACSL1 inhibitor or a pharmaceutically acceptable salt thereof in an effective amount. In an embodiment, the ACSL1 inhibitor may be administered to the subject before, during, or after consumption of a high fat food.

These and other features of the present subject matter will become readily apparent upon further review of the following specification.

### BRIEF DESCRIPTION OF THE DRAWINGS

FIG. 1 shows a schematic of the ACSL pathway and the place of action of an ACSL inhibitor.

FIGS. 2(A) and 2(B) show the results of studies demonstrating the effects of an exemplary ACSL1 inhibitor (Triacsin C) treatment on lipid accumulation in macrophage cells in response to an inflammation cytokine. FIG. 2A shows cell culture lipid droplet accumulation (red) in cells in culture; FIG. 2B shows the lipid accumulation in cells determined by flow cytometry (FACS) analysis.

FIGS. 3(A), 3(B), and 3(C) show protein or gene expression levels for inflammation or infiltration markers IL-B protein; TNF- a mRNA and CD1lb mRNA, respectively for macrophage cells treated with the exemplary ACSL1 inhibitor.

FIG. 4(A) shows a graph of ACSL1 gene expression in macrophages exposed to a control or to palmitic acid for 4 hours.

FIG. 4(B) shows a graph of ACSL1 protein expression in M\macrophages exposed to a control or to palmitic acid for 4 hours.

FIG. 4(C) shows a graph of monocyte infiltration and inflammatory marker gene expression in the presence or absence of 30-minute pre-treatment with Triacsin C (ACSL1 Inhibitor) before palmitic acid exposure.

FIG. 4(D) shows a graph of further monocyte infiltration and inflammatory marker gene expression in the presence or absence of 30-minute pre-treatment with Triacsin C (ACSL1 Inhibitor) before palmitic acid exposure.

FIG. 5 shows the effect of Triacsin C pre-treatment on macrophage expression of fatty acid uptake related genes in response to palmitic acid.

FIG. 6(A) shows the effect of siRNA ACSL1 silencing on ACSL1 gene expression.

FIG. 6(B) shows the effect of siRNA ACSL1 silencing on ACSL1 protein expression.

FIG. 6(C) shows the effect of siRNA ACSL1 silencing on macrophage gene expression of monocyte inflammatory and infiltration markers in response to palmitic acid exposure.

FIG. 7(A) shows a flow cytometry plot illustrating the ability of treatment of mice with drinkable Triacsin C 30 minutes prior to introducing the mice to a high fat food to inhibit the inflammatory response in monocytes (as measured by the number of CD45+, Ly6C+, and CD11b+ cells).

FIG. 7(B) shows a graph of flow cytometry data collected during an experiment measuring the ability of treatment of mice with drinkable Triacsin C 30 minutes prior to introducing the mice to a high fat food to inhibit the inflammatory response in monocytes (as measured by the number of CD45+, Ly6C+, and CD11b+ cells).

FIG. 8 shows a flow cytometry plot illustrating the ability of treatment of mice fed a high fat diet with a low dose of drinkable Triacsin C to inhibit the inflammatory response in monocytes (as measured by the number of CD45+, Ly6C+, and CD11b+ cells).

FIG. 9(A) depicts a model of an experiment testing treatment of mice with Triacsin C to prevent weight gain upon consumption of high fat diet.

FIG. 9(B) depicts a graph of mouse body weight changes over 4 weeks of high fat diet with or without Triacsin C treatment.

FIG. 9(C) depicts a graph of the level of inflammatory monocytes found circulating in blood stream of mice over 4 weeks of high fat diet with or without Triacsin C treatment.

FIG. 10 depicts the effect of 4 weeks of a high fat diet on mice with or without Triacsin C treatment on liver size, visceral and subcutaneous fat.

Similar reference characters denote corresponding features consistently throughout the attached drawings.

### DETAILED DESCRIPTION OF THE PREFERRED EMBODIMENTS

### Definitions

As used herein, an “effective” amount may refer to a therapeutically effective amount for the prevention and/or inhibition of postprandial increase in inflammatory markers or monocyte polarization to pro-inflammatory macrophages. Inflammatory markers may include, for example, C-reactive proteins (CRP); cytokines, such as TNF-α, interleukins, such as IL-6; or adhesion molecules, such as VCAM or ICAM, GDF-15 or ST2. In the alternative, an “effective” amount may refer to a therapeutically effective amount for the prevention and/or treatment of weight gain or a disease associated with consumption of a high fat diet, such as obesity, diabetes, arthritis, hepatic steatosis (fatty liver) and the like.

As used herein, the term “subject” may refer to a mammal such as a human.

As used herein, the term “about” when used to modify a numeral, means within 10% of the numeral's value.

As used herein, “Triacsin C” refers to an inhibitor of long fatty acyl CoA synthetase (chemical formula C₁₁H₁₇N₃O; chemical name N-(((2E,4E,7E)-undeca-2,4,7-trienylidene)amino)nitrous amide) that may be isolated from Streptomyces aureofaciens. Triacsin C may also be isolated from fungi as a fungal metabolite.

As used herein, “hepatic steatosis” refers to a medical condition characterized by an increased buildup of fat in the liver. This condition may also be called “liver steatosis” or “fatty liver disease”.

As used herein, “dyslipidemia” refers to a medical condition characterized by an abnormally elevated level of cholesterol or fat in the blood stream. Dyslipidemia may increase the risk of developing atherosclerosis, heart attack, and stroke.

As used herein, “obesity” refers to a medical condition characterized by having a body mass index (BMI) of 30 or greater. Obesity may increase the risk of developing heart disease, diabetes, high blood pressure, and certain cancers.

Salts encompassed within the term “pharmaceutically acceptable salts” as used herein refer to non-toxic salts of the ACSL1 inhibitors which are generally prepared by reacting a free base with a suitable organic or inorganic acid or by reacting an acid with a suitable organic or inorganic base. Particular mention may be made of the pharmaceutically acceptable inorganic and organic acids customarily used in pharmacy. Those suitable are in particular water-soluble and water-insoluble acid addition salts with acids such as, for example, hydrochloric acid, hydrobromic acid, phosphoric acid, nitric acid, sulfuric acid, acetic acid, citric acid, D-gluconic acid, benzoic acid, 2-(4-hydroxybenzoyl)-benzoic acid, butyric acid, sulfosalicylic acid, maleic acid, lauric acid, malic acid, fumaric acid, succinic acid, oxalic acid, tartaric acid, embonic acid, stearic acid, toluenesulfonic acid, methanesulfonic acid or 1-hydroxy-2-naphthoic acid. As examples of pharmaceutically acceptable salts with bases may be mentioned the lithium, sodium, potassium, calcium, aluminum, magnesium, titanium, ammonium, meglumine or guanidinium salts.

### Formulations and Methods

The formulation for treating or inhibiting postprandial inflammation according to the present disclosure includes a composition comprising an ACSL1 inhibitor or a pharmaceutically acceptable salt thereof In an embodiment, the composition is suitable for oral administration to a subject. The composition may be edible, such as a beverage or soluble additive to a beverage, and the beverage may be a carbonated beverage. In an embodiment, the ACSL1 inhibitor is a known inhibitor, such as Triacsin C, 2-Fluoropalmitic acid or Adenosine 5′-hexadecylphosphate (AMPC 16).

In another embodiment, the present subject matter relates to a method of treating inflammation comprising administering to a subject in need thereof a composition comprising an ACSL1 inhibitor or a pharmaceutically acceptable salt thereof within a time frame of hours of the subject consuming a food. The time frame may be before, during or after consuming the food. In an embodiment, the composition is administered orally, for example, in the form of a beverage or soluble additive to a beverage. In an embodiment, the composition is administered by consumption by the subject at about the same time as the subject consumes the food, namely before, during or after the subject consumes the food. The food may be high in SFA. The subject may be afflicted with any condition(s) characterized with elevated TNF-α. Such conditions may include, without limitation, obesity, diabetes and arthritis. The subject may be an obese person, or otherwise a person with excessive adipose tissue.

In another embodiment, the present subject matter relates to a method of preventing weight gain comprising administering to a subject in need thereof an effective dose of a composition comprising the ACSL1 inhibitor or a pharmaceutically acceptable salt thereof within a time frame of hours of the subject consuming a food. In an embodiment, the composition is administered orally, for example, in the form of a beverage or soluble additive to a beverage. The time frame may be before, during or after consuming the food. In an embodiment, the composition is administered by consumption by the subject at about the same time as the subject consumes the food, namely immediately before, during or immediately after the subject consumes the food. The food may be a high fat food. The fat in the high fat food may be SFA. The subject may be afflicted with or at risk of developing any condition(s) characterized with elevated TNF-α. Such conditions may include, without limitation, obesity, diabetes and arthritis. The subject may be an obese person, or otherwise a person with excessive adipose tissue.

In another embodiment, the present subject matter relates to a method of preventing development or progression of a disease associated with eating a high fat food. For example, the method may prevent the development or progression of dyslipidemia, obesity, diabetes, arthritis, hepatic steatosis (fatty liver) and the like in a subject. The method may comprise administering to a subject in need thereof an effective dose of a composition comprising an ACSL1 inhibitor or a pharmaceutically acceptable salt thereof within a time frame of hours of the subject consuming a food. In an embodiment, the composition is administered orally, for example, in the form of a beverage or soluble additive to a beverage. The time frame may be before, during or after consuming the food. In an embodiment, the composition is administered by consumption by the subject at about the same time as the subject consumes the food, namely immediately before, during or immediately after the subject consumes the food. The food may be a high fat food. The fat in the high fat food may be SFA. The subject may be afflicted with or at risk of developing any condition(s) characterized with elevated TNF-α. Such conditions may include, without limitation, obesity, diabetes and arthritis. The subject may be an obese person, or otherwise a person with excessive adipose tissue. In certain non-limiting embodiments, the subject may be an obese person who is at further risk of developing diabetes or arthritis.

In an embodiment, any of the present methods may include administering an ACSL1 inhibitor or a pharmaceutically acceptable salt thereof to a subject in need thereof before the subject's consumption of a food. In a further embodiment, the administration of the ACSL1 inhibitor may be 4 hours, 3 hours, 2 hours, 1 hour, 30 minutes, 20 minutes, 10 minutes, 5 minutes, or 1 minute before the consumption of the food. In certain embodiments, the administration of the ACSL1 inhibitor may be 30 minutes before the consumption of the food. In other embodiments, the administration of the ACSL1 inhibitor may be 3 hours before the consumption of the food.

In another embodiment, any of the present methods may include administering an ACSL1 inhibitor or a pharmaceutically acceptable salt thereof to a subject in need thereof daily. In a further such embodiment, the administration of the ACSL1 inhibitor may be 1, 2, or 3 times daily. In certain embodiments, the ACSL1 inhibitor may be administered to a fasted or fed subject.

In an embodiment, the present methods may include administering an ACSL1 inhibitor or a pharmaceutically acceptable salt thereof to a subject in need thereof in order to reduce the expression of ACSL1. The reduced expression of ACSL1 may in turn reduce the normal upregulation of monocytic inflammatory markers and infiltration markers in response to consumption of a high fat food. Further, the ACSL1 inhibitor may result in a reduction of fat accumulation in macrophages, and may thereby reduce both the inflammatory response to high fat foods and prevent weight gain and diseases and conditions associated with weight gain. In an embodiment, the ACSL1 inhibitor may be Triacsin C.

The present composition can act as a neutralizer to acute effects of fatty acids ingested by the subject during consumption of foods, such as fast foods, rich in fatty acids such as SFA, thereby preventing or minimizing the acute effects of said SFA. After consumption, fatty acids become emulsified in the duodenum, where each three fatty acids get packaged with a glycerol group to produce “triglycerides”. These triglycerides circulate throughout the body via apolipoproteins to distribute energy and essential fatty acids needed for other processes. An edible form of an ACSL inhibitor can prevent the formation of triglycerides rich in long chain fatty acids, such as palmitic acid (PA). Accordingly, in an embodiment the present formulations and compositions are formulated for administration as an oral dosage form. Non-limiting examples of such oral dosage forms include a beverage, a soluble additive to a beverage, and an edible candy or snack, including but not limited to edible gummies.

Alternatively, a direct injection of an ACSL inhibitor into inflamed adipose tissue could also be used to reduce inflammation at the site. ACSL1 inhibitors useful herein may be any compound that inhibits ACSL1, such as, by way of non-limiting examples, Triacsin C, 2-Fluoropalmitic acid or AMPC16.

In addition to the ACSL inhibitor, the present compositions may include at least one additional ingredient, including but not limited to caffeine.

### ACSL Target Pathway

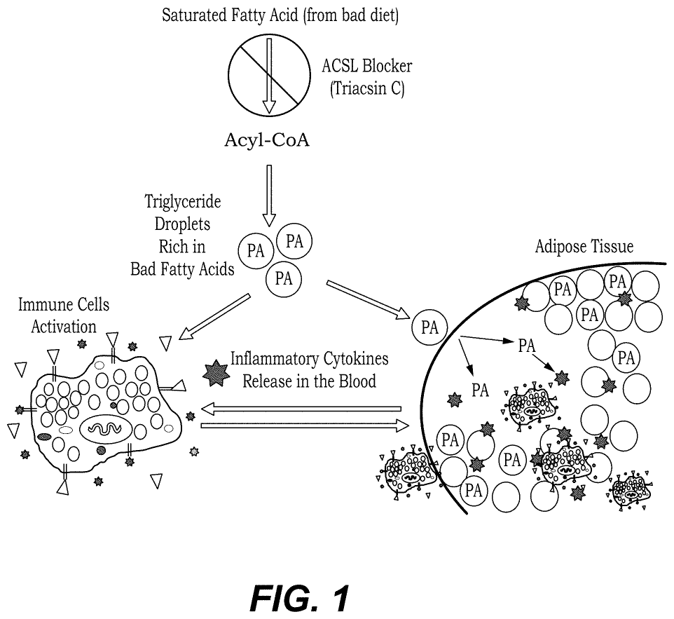

FIG. 1 shows a proposed schematic of the ACSL target pathway. Free fatty acids can only be utilized by the body after their activation to become Acyl-CoA. This process is catalyzed by Acyl-CoA synthetase (ACSL). Even though dietary fatty acids are essential for several metabolic processes, SFA such as PA induce potentially harmful or otherwise unwanted responses, such as macrophage foaming and pro-inflammatory responses. Blocking bad fatty acid activation through administration of an ACSL inhibitor shortly before, during or shortly after high intake of free fatty acids can stop the accumulation of triglyceride droplets in the blood, which could trigger an immune response if present, thereby potentially preventing further pro-inflammatory response and damage. Additionally, Acyl-CoA activation is important for TNF-α signaling, and the use of ACSL inhibitors can stop TNF-α signals, thus stopping inflammatory responses. Therefore, any condition with elevated TNF-α should benefit from such an inhibitor. In particular, a postprandial acute inflammatory response should benefit from such an inhibitor, specifically administered soon before, at the same time as, or soon after eating.

In particular, ACSL1 inhibitors are shown in the following examples to prevent monocyte polarization to pro-inflammatory macrophages, to prevent fat accumulation in macrophages, and to prevent weight gain upon consumption of a high fat diet.

### EXAMPLE 1

### Materials and Methodologies

Cell culture: Human monocytic leukemia cell line (THP-1) cells were purchased from American Type Culture Collection (ATCC). Cell cultures were maintained in RPMI-1640 culture medium (Gibco, Life Technologies, Grand Island, USA) supplemented with 10% fetal bovine serum (Gibco, Life Technologies, Grand Island, N.Y., USA), 2 mM glutamine (Gibco, Invitrogen, Grand Island, N.Y., USA), 1 mM sodium pyruvate, 10 mM HEPES, 100 ug/m1 Normocin 50 U/ml penicillin and 50 μg/m1 streptomycin (P/S; Gibco, Invitrogen, Grand Island, N.Y., USA) and incubated at 37° C. (with humidity) in 5% CO2.

Cell stimulation: Prior to stimulation, THP-1 cells were plated in 24-well plates (Costar, Corning Incorporated, Corning, N.Y., USA) at 5×105 cells/well cell density unless indicated otherwise. Cells were incubated with either Triacsin C, a long chain acyl-CoA synthetase (ACSL1) inhibitor or Etomoxir, a carnitine palmitoyltransferase-1 (CPT-1) inhibitor (CPT-1 is a mitochondrial enzyme involved in fatty acid (3-oxidation). Etomoxir is an irreversible inhibitor of carnitine palmitoyltransferase-1 (CPT-1), and is used widely as a small-molecule inhibitor of fatty acid oxidation (FAO). FAO is the process by which active fatty acids stored in triglycerides are released when energy is needed. Blocking FAO stops fatty acids from being liberated and released from adipose cells. Etomoxir acts as a positive control, in that it acts oppositely from ACSL. ACSL inhibitors prevents fat from going into the cells (fatty acid depletion), while Etomoxir prevent fat from being burned (fatty acid accumulation). A control of no treatment, also referred to as vehicle, was also used in the experiments.

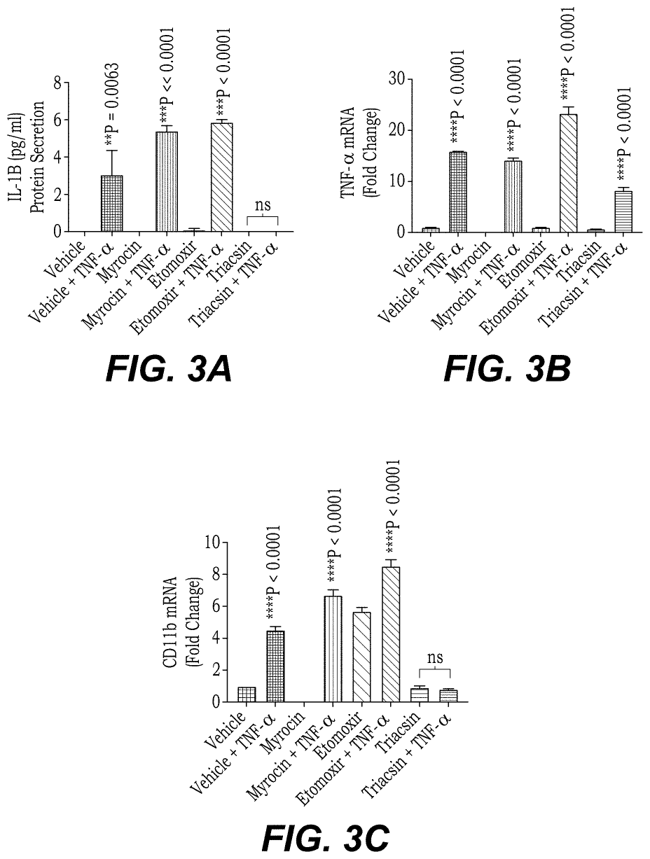

All lipid inhibitors were purchased from Invivogen (InvivoGen, San Diego, Calif., USA). Cultures were then stimulated with TNF-α (10 ng/ml) overnight at 37° C. Cells were harvested for cell sorting (FACS) analysis, RNA isolation for gene expression and conditioned media were collected for measuring IL-1β secretion levels. IL-1β is a pro-inflammatory cytokine indicative of inflammation by secretion level (see ELISA results in FIG. 3A). Exemplary RNA gene expression results are shown for, e.g., TNF-α (FIG. 3B) and other monocyte inflammatory and infiltration markers, such as CD11b (FIG. 3C) gene regulators. Conditioned media were collected and stored at −80° C.

Macrophage transformation: Prior to macrophage transformation of THP-1 monocytes, cells were plated at 106 cells per ml in 24 well plates (unless specified otherwise) and pre-treated with different lipid metabolite inhibitors (Triacsin C or Etomoxir, as above) or media alone as the vehicle condition for 1 hour at 37° C. The cells being treated as above were then further treated with 10 ng/ml phorbol 12-myristate 13-acetate (PMA, Sigma) for 24 hours at 37° C. to facilitate differentiation into macrophages. The monocyte-derived macrophages were washed three times with phosphate-buffered saline (PBS) and further incubated for 48 h in serum-free RPMI (37° C., 5% CO2). Resulting cultures were subject to the assays mentioned below and data were collected for the different analysis.

Extracellular staining flow cytometry: After experimental or control treatments, the macrophages were lifted by using phosphate-buffered saline (PBS) supplemented with ethylenediaminetetraacetic acid (EDTA). Collected cells were then centrifuged and washed 3 times. The monocytic cells (1×10⁶ cells/mL) were resuspended in FACS staining buffer (BD Biosciences) and blocked with human IgG (Sigma; 20 μg) for 30 minutes on ice. Cells were washed three times with FACS buffer and resuspended in 2% paraformaldehyde. Cells were centrifuged and resuspended in FACS buffer for FACS analysis (FACSCanto II; BD Bioscience, San Jose, USA). FACS data analysis was performed using BD FACSDiva™ Software 8 (BD Biosciences, San Jose, USA). Cells were sorted according to their size and granulation using the protocol presented by Lee et al., 2004 for lipid measurement (Lee, Y. H., Chen, S. Y., Wiesner, R. J., & Huang, Y. F. (2004). Simple flow cytometric method used to assess lipid accumulation in fat cells. Journal of lipid research, 45(6), 1162-1167), the content of which is incorporated herein.

Oil Red O staining: To determine the degree of lipid accumulation, the accumulation of cytoplasmic triglycerides in cells were measured by staining with Oil Red O (SigmaAldrich, St Louis, Mo., USA). Briefly, the cells were washed three times in PBS, then fixed with 4% paraformaldehyde for 30 min at room temperature. The cells were incubated with 60% isopropanol for 5 min, then washed with deionized water. The cells were incubated with 0.5% Oil Red O solution for 5 min to label lipid droplets. After staining, the cells were washed several times with deionized water to remove excess stain, then counterstained with hemotoxylin to visualize general structure. The stained cells were photographed at ×40 magnification using a Zeiss Axio Inverted Microscope.

### EXAMPLE 2

### Results

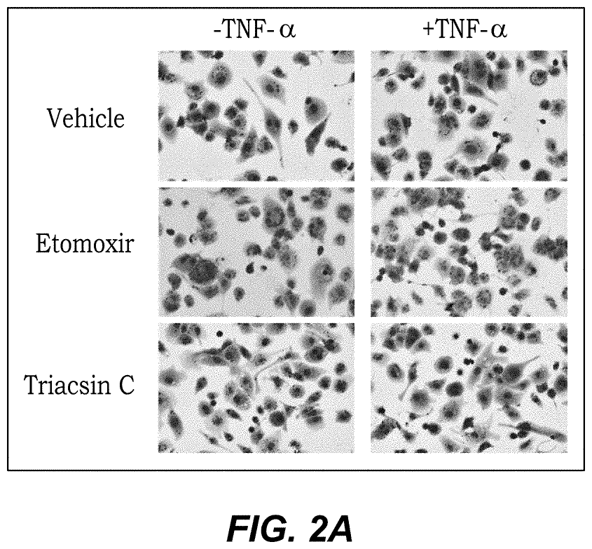

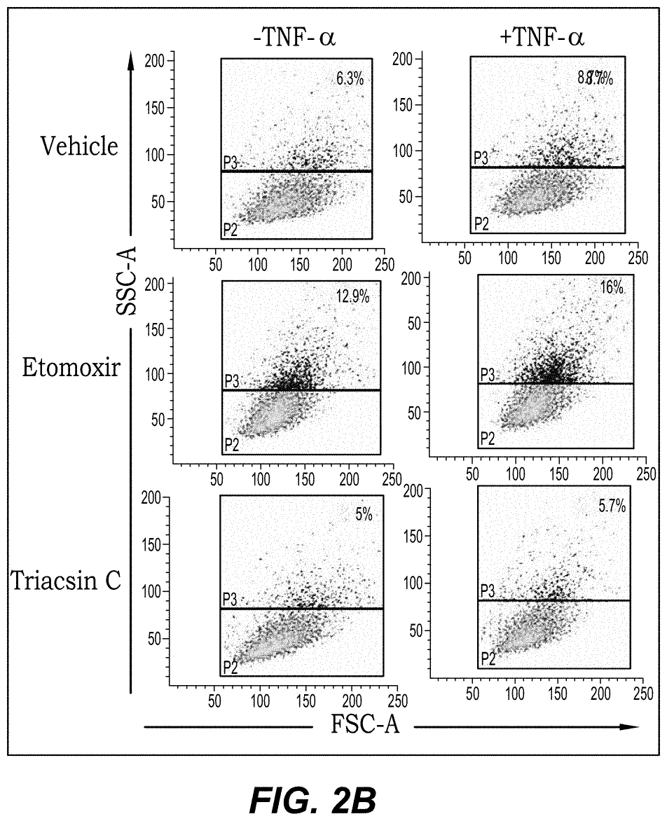

Results of the Oil red O staining show Triacsin C prevents lipid accumulation in response to TNF-a, particularly relative to positive controls of vehicle and Etomoxir treatments, alone (FIG. 2A). These results confirm the findings indicated in the FACS analysis shown in FIG. 2B. Treated cultured cells were visualized under a microscope. Red color in FIG. 2A represents fat/ lipid accumulation within the macrophages.

FIG. 2B shows dot plots of the results from the previously described FACS analysis. Both the size of the cells and their granulation are used to assess lipid accumulation. Green dots represent the general population size and blue dots represent those that accumulated fat. Both vehicle treated and Etomoxir treated cells showed increased lipid accumulation in response to the presence of TNF-α (percentages in top right of plots). In contrast, the exemplary ASCL-1 inhibitor (Triacsin C) treated cells exhibited no significant change in apparent lipid accumulation.

FIG. 3A shows ELISA measurements of protein expression levels of pro inflammatory cytokine IL-1B and gene expression (mRNA) levels of TNF-α and another inflammation marker CD11b, respectively, in macrophage cells treated as above. Only Triacsin C treated cells showed little to no change in inflammatory markers in response to TNF-α treatment. Thus, treatment with Triacsin C not only reduces or prevents accumulation of lipids in macrophages, but reduces or prevents a subsequent inflammatory response.

### EXAMPLE 3

### Effect of Triacsin C on the Inflammatory Response of Monocytes/Macrophages to a High Fat Diet

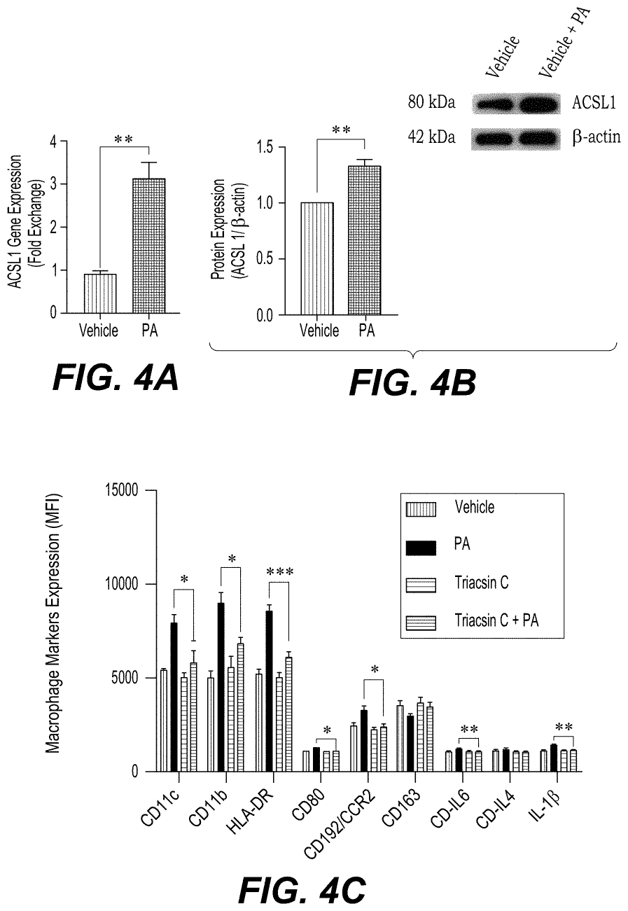

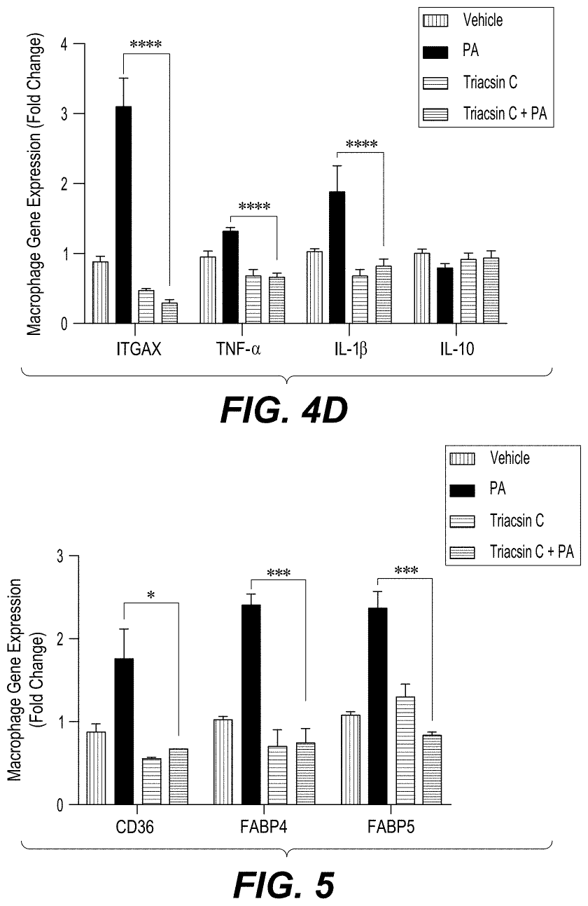

Experiments were conducted on human macrophages fed either a control vehicle or palmitic acid for four hours to confirm that exposure to palmitic acid normally increases ACSL1 gene expression and protein expression (see FIGS. 4(A)-4(B)). Palmitic acid induced upregulation of ACSL1, suggesting that ACSL1 is involved in the process of metabolizing palmitic acid. Pre-treatment of human macrophages with Triacsin C (30 minutes treatment before addition of palmitic acid) was shown to reduce the upregulation of monocytic inflammatory markers (IL-1b, CD11c, HLA-DR) and to reduce infiltration markers (CD11b, CCR2, and CD80), which would make the circulating inflamed monocytes/macrophages less likely to adhere to tissues and organs (See FIGS. 4(C)-4(D)).

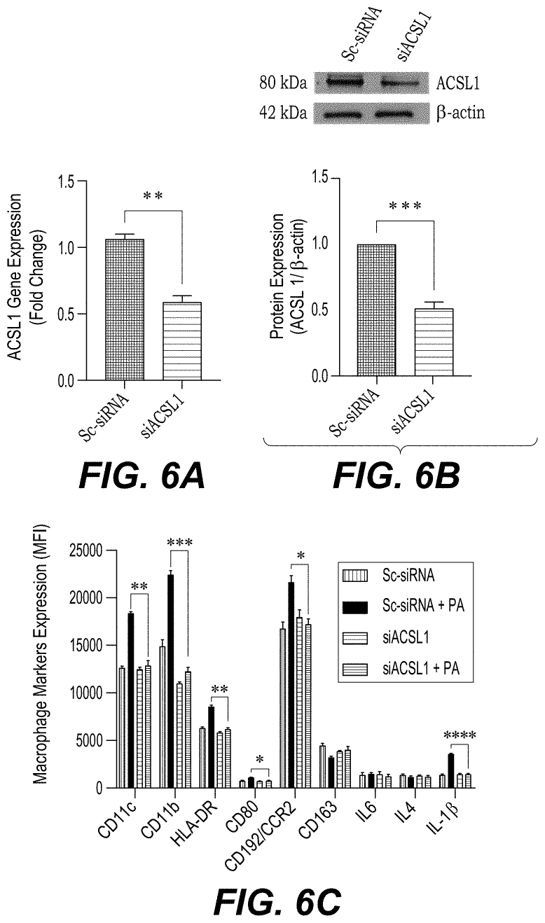

A further investigation demonstrated that blocking the ACSL enzyme inhibited expression of fatty acid uptake-related genes (FIG. 5) which in turn reduced the amount of intracellular fat found within monocytes (data not shown). These findings were verified by genetically silencing the ACSL1 isozyme using siRNA targeting ACSL1 followed by repeated feedings of palmitic acid. The resulting reduction in ACSL1 gene expression, protein expression, and monocytic inflammatory and infiltration markers mirrored that found using Triacsin C treatment (FIGS. 6(A)-6(C)).

### EXAMPLE 4

### Effect of Triacsin C on HFD-Induced Inflammation and Weight Gain in Mice

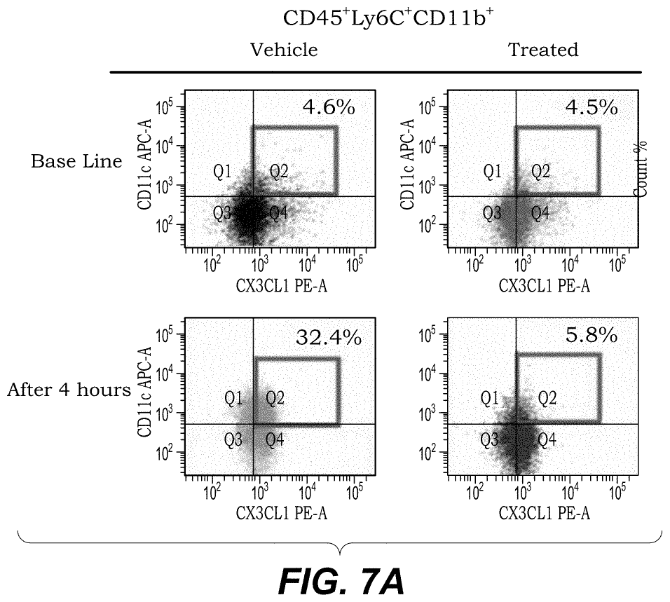

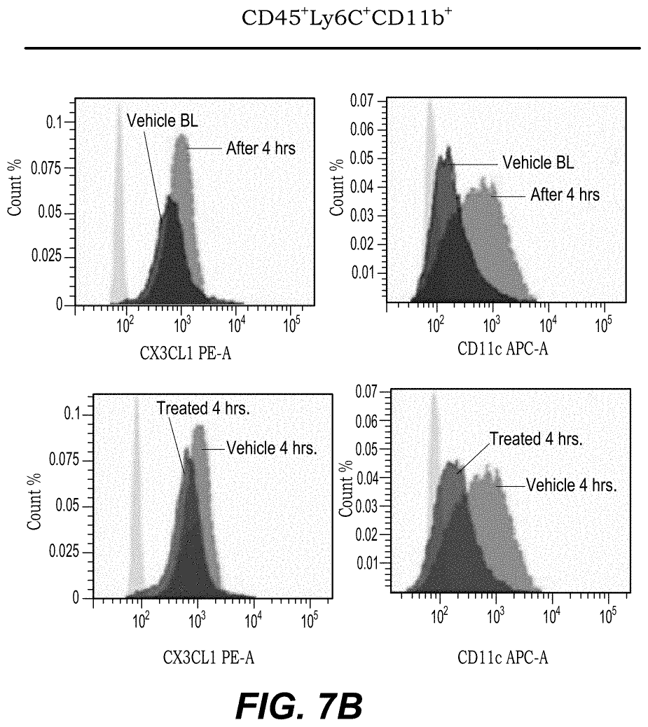

Further experiments were developed to test the utility of Triacsin C as an anti-inflammatory and/or weight gain prevention drug in a mouse model. Base line blood samples were taken from each mouse before feeding. Mice were then administered either Triacsin C (10 mg/m1) or a vehicle (control) in a drinkable form 30 minutes prior to introducing them to high fat food. Four hours after food consumption another blood sample was collected from each mouse and the inflammatory response was measured using flow cytometry to look for pro-inflammatory monocytes (CD45⁺/Ly6C/CD11b⁺ cells). (See FIGS. 7(A)-7(B)) The experiment was repeated 5 times on non-consecutive days and also confirmed that the dose was safe.

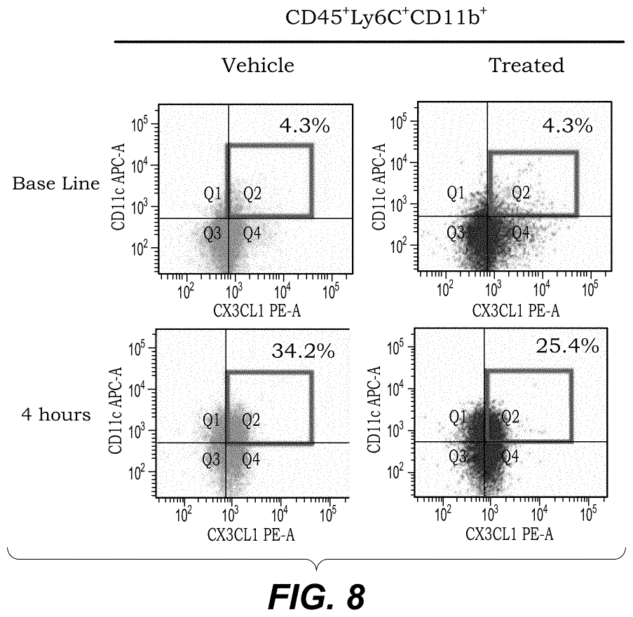

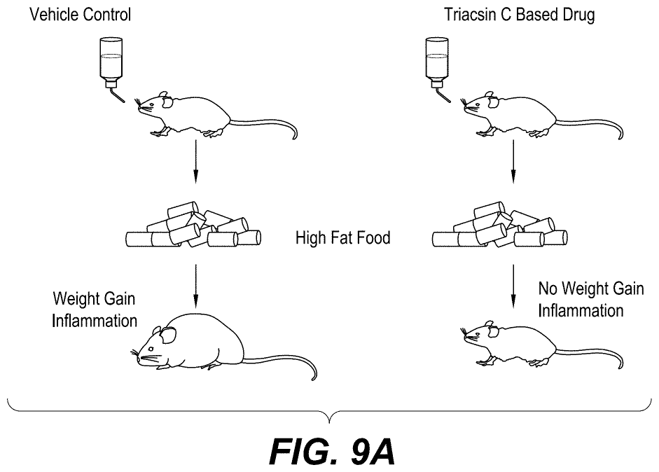

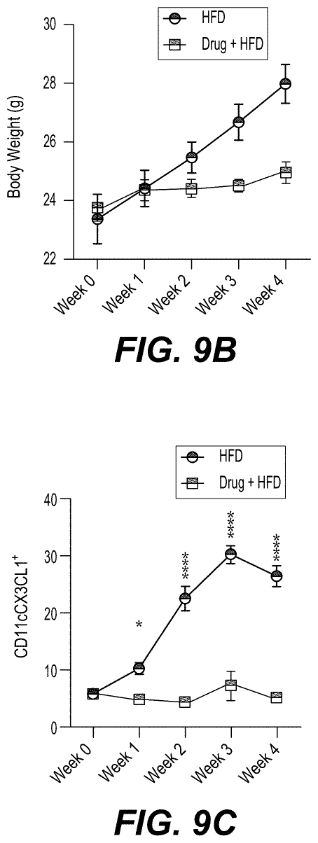

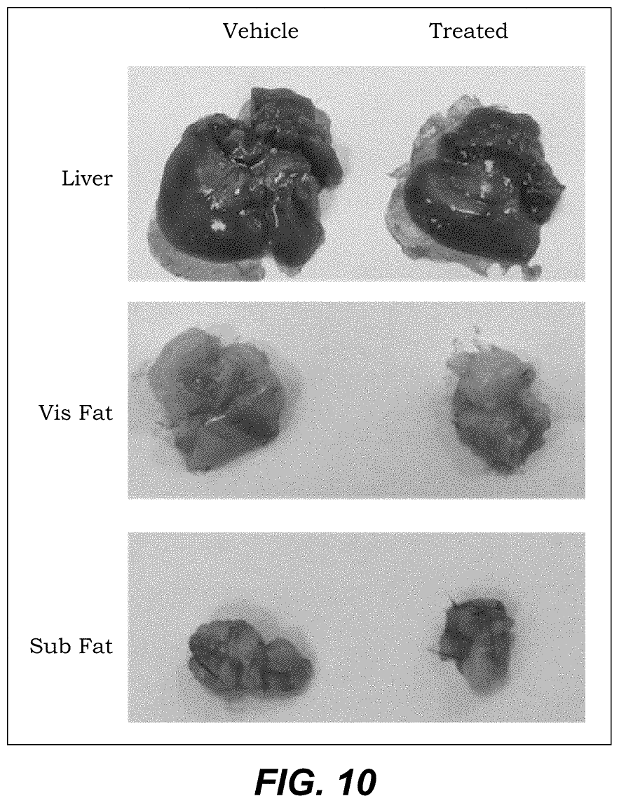

A further experiment was designed to test whether constant daily doses of Triacsin C could prevent weight gain (or the development of obesity and other weight gain related diseases). An initial experiment was also conducted to test the effect of this low dose on acute high fat diet feeding (a four-hour feeding experiment akin to the design described above) and the lower dose of Triacsin C was demonstrated to reduce the inflammatory upregulation by 10% (pro-inflammatory monocytes reduced from 34.2% in the control to 25.4% in the treatment group, See FIG. 8) Then a low dose sufficient to block fatty acid uptake was calculated at 0.05 mg/kg. Mice were placed on a constant high fat diet (HFD) for up to 5 weeks in the presence of a daily (drinkable) dose of either Triacsin C or the vehicle control (FIG. 9(A)). This experiment demonstrated that weight gain resulting from HFD was dramatically prevented by administration of a low daily dose of Triacsin C (FIG. 9(B)). Further, this experiment demonstrated that administration of a low daily dose of Triacsin C prevented the increase in inflammatory monocytes observed to result from long term exposure to a HFD (FIG. 9(C)). Finally, the livers of mice fed a HFD in the presence of a daily dose of Triacsin C were smaller, and had significantly less adipose tissue (fat) than the livers of mice fed the vehicle control (FIG. 10).

In conclusion, these experiments demonstrate that daily administration of Triacsin C can prevent weight gain, inflammatory response, and progression/development of diseases associated with weight gain (such as hepatic steatosis).

It is to be understood that the formulation and method of treating postprandial inflammation and preventing weight gain is not limited to the specific embodiments described above, but encompasses any and all embodiments within the scope of the generic language of the following claims enabled by the embodiments described herein, or otherwise shown in the drawings or described above in terms sufficient to enable one of ordinary skill in the art to make and use the claimed subject matter.

We claim:

1. A method of inhibiting an inflammatory response in a subject, comprising administering an ACSL1 inhibitor or a pharmaceutically acceptable salt thereof to a subject in need thereof in an amount effective to inhibit an increase in inflammatory markers or monocyte polarization to pro-inflammatory macrophages resulting from the subject's consumption of a food that is high in at least one saturated fatty acid.

2. The method of claim 1, wherein the ACSL1 inhibitor is selected from the group consisting of Triacsin C, 2-Fluoropalmitic acid and Adenosine 5′-hexadecylphosphate.

3. The method of claim 2, wherein the ACSL1 inhibitor is Triacsin C.

4. The method of claim 3 wherein the subject has one or more of obesity, insulin resistance, diabetes and arthritis.

5. The method of claim 1, wherein the ACSL1 inhibitor is administered orally.

6. The method of claim 1, wherein the ACSL1 inhibitor is administered in a beverage.

7. The method of claim 6, wherein the beverage is a carbonated beverage.

8. The method of claim 1, wherein the ACSL1 inhibitor is administered within three hours before the subject's consumption of the food.

9. The method of claim 8, wherein the ACSL1 inhibitor is administered within thirty minutes before the subject's consumption of the food.

10. The method of claim 1, wherein the at least one saturated fatty acid is palmitic acid.

11. A method of preventing weight gain in a subject, comprising administering a daily dose of an effective amount of an ACSL1 inhibitor or a pharmaceutically acceptable salt thereof to a subject in need thereof.

12. The method of claim 11, wherein the ACSL1 inhibitor is selected from the group consisting of Triacsin C, 2-Fluoropalmitic acid and Adenosine 5′-hexadecylphosphate.

13. The method of claim 12, wherein the ACSL1 inhibitor is Triacsin C.

14. The method of claim 11, wherein the ACSL1 inhibitor is administered orally.

15. The method of claim 11, wherein the ACSL1 inhibitor is administered in either a carbonated beverage or an edible gummy.

16. A method of preventing the development or progression of a disease associated with consumption of high fat foods in a subject, comprising administering a daily dose of an effective amount of an ACSL1 inhibitor or a pharmaceutically acceptable salt thereof to a subject in need thereof.

17. The method of claim 16, wherein the disease is selected from the group consisting of dyslipidemia, obesity, diabetes, arthritis, and hepatic steatosis.

18. The method of claim 16, wherein the ACSL1 inhibitor is selected from the group consisting of Triacsin C, 2-Fluoropalmitic acid and Adenosine 5′-hexadecylphosphate.

19. The method of claim 16, wherein the ACSL1 inhibitor is Triacsin C.

20. The method of claim 16, wherein the ACSL1 inhibitor is administered in either a carbonated beverage or an edible gummy.
